(19) United States
(12) Patent Application Publication
(10) Pub. No.: US 2004/0137590 A1
(43) Pub. Date: Jul. 15, 2004

(54) Allene oxide cyclase gene and use thereof for producing jasmonic acid

(76) Inventors: J?ouml;rg Ziegler, Halle/Saale (DE); Irene Stenzel, Halle/Saale (DE); Bettina Hause, Halle/Saale (DE); Claus Wasternack, Halle/Saale (DE)

Correspondence Address: NEEDLE & ROSENBERG, P.C., SUITE 1000, 999 PEACHTREE STREET, ATLANTA, GA, 30309-3915, US

(21) Appl. No.: 10/203,064

(22) PCT Filed: Feb. 2, 2001
(86) PCT No.: PCT/EP01/01148

(30) Foreign Application Priority Data
Feb. 2, 2000 (DE) 100 04 468.9

(51) Int. Cl. C07H 21/04; C12N 9/02; C12N 5/04

(52) U.S. Cl. 435/189; 435/69.1; 435/320.1; 435/419; 536/23.2

(57) ABSTRACT

The present invention concerns a nucleic acid that codes for a plant allene oxide cyclase (AOC). The provision of the new cDNA clone now permits for the first time the production of jasmonic acid in large quantities and of great purity by biotechnological means.

The present invention concerns a nucleic acid that codes for a plant allene oxide cyclase and the use of nucleic acid for the production of jasmonic acid by biotechnological means.

Jasmonic acid (JA) and its methyl esters (JAME), which are together also referred to as jasmonates, consist of a cyclopentanone ring, to which an acetic acid and a pentenyl side chain is added. These side chains are present either in the cis (3R/7S) or trans form (3R/7R). In addition, a large number of structurally related compounds have been described that are commonly found in plants (Hamberg, M. and Gardner, H. W. (1992) Biochem. Biophys. Acta 1165, 1-18). The first physiological function found for JA was inhibition of plant growth, found in 1971 (Aldridge, D. C., et al. (1971) J. Chem. Soc. Chem. Commun. 1623 1627). From then on, jasmonates were regarded as the signals of a modified gene expression in various plants as a response to biotic and abiotic stress and also as a signal of specific developmental processes during plant development (Creelman, R. A. and Mullet, J. E. (1997) Annu. Rev. Plant Physiol. Plant Mol. Biol. 48, 355-381; Wasternack, C. and Parthier, B. (1997) Trends in Plant Science 2, 302-307). In 1990, it was found by Farmer and Ryan (Farmer, E. E. and Ryan, C. A. (1990) Proc. Natl. Acad. Sci. USA 87, 7713-7716) that JA is an important intermediate product in wound-induced signal cascade as a result of attack by plant eaters on tomato leaves. Jasmonate was identified as a trigger for many defence mechanisms in plants in the presence of a pathogenic infection or abiotic stress. Besides the expression of defence genes, such as proteinase inhibitors, the syntheses of phytoalexins, alkaloids and smell attractants are the most important responses to JA, which in most cases takes places via the up-regulation of specific enzymes (Ellard-Ivey, M. and Douglas C. J. (1996) Plant Physiol. 112, 183-192; Feussner, I. et al. (1995) Plant J. 7, 949-957). The responses to jasmonate were characterised via a modified expression of specific genes, via jasmonate-insensitive and jasmonate defect mutants, via transgenic plants with a jasmonate deficiency or via an increase in endogenous jasmonates and via inhibition studies. Only recently was the JA precursor 12-oxo-phytodienic acid (OPDA) regarded as a preferred signal of a JA response. During the developmental processes, pollen maturation and seedling growth are JA-dependent.

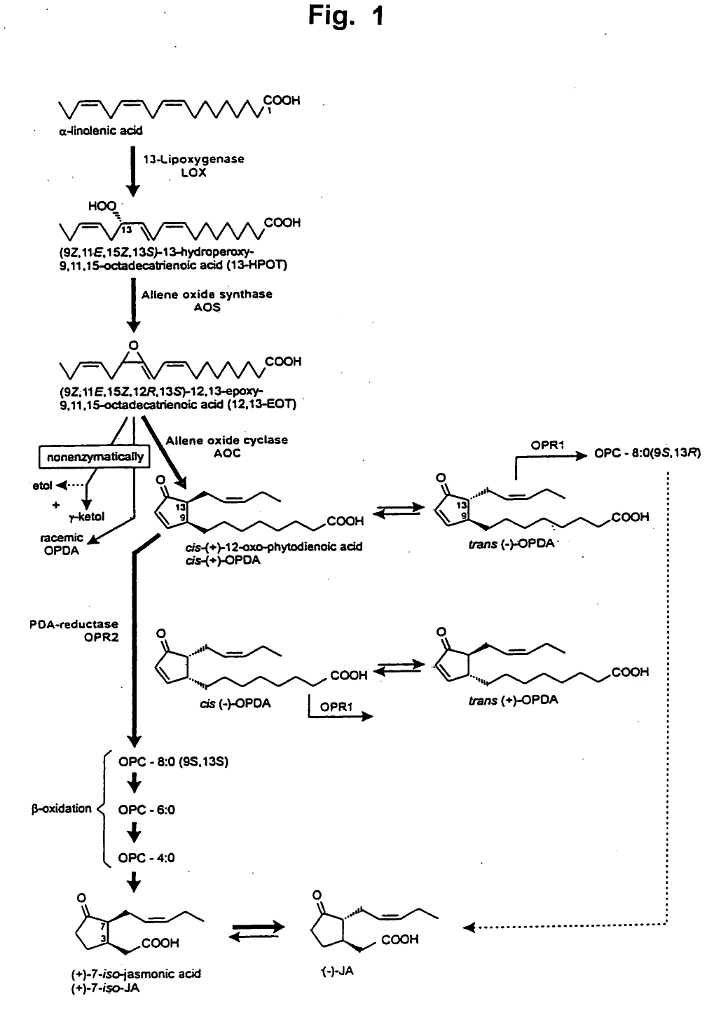

The biosynthesis of JA takes place via the oxylipin biosynthesis pathway, which starts with the incorporation of molecular oxygen in position 13 of linolenic acid that is catalysed by a lipoxygenase. The fatty acid hydroperoxide formed (13(S)-hydroperoxy-9(Z),11(E),15(Z)-octadecatrienic acid,13-HPOT) is then dehydrated by allene oxide synthase (AOS) to form an allene oxide (Hamberg, M. and Gardner, H. W. (1992) Biochem. Biophys. Acta 1165,1-18). This allene oxide is then cyclised by allene oxide cyclase (AOC) to form 9(S), 13(S)-OPDA. Following reduction of the double bond in the ring, catalysed by a reductase and three following rounds of β oxidation, (+)-7-iso-JA is formed, e.g. 3(R), 7(S)-JA. The authors Vick and Zimmermann proposed a similar biosynthesis pathway back in 1983, but it was assumed that the formation of OPDA is catalysed from 13-HPOT from a single enzyme, a so-called hydroperoxide cyclase (Vick, B. A. and Zimmermann, D. C. (1983) Biochem. Biophys. Res. Commun. 111, 470-477). In 1988, Hamberg showed that this step is performed by 2 enzyme activities. One of these enzymes, which displays a membrane-bound activity and was later characterised as AOS, catalyses the formation of an unstable allene oxide, which is rapidly broken down with a half-life of 25 seconds by chemical hydrolysis to form α- and γ-ketol and racemic OPDA (FIG. 1). In this connection, OPDA accounts for only 10-15% of the total quantity of the degradation products and is a racemic mixture consisting of the cis isomers 9(S),13(S) and 9(R),13(R).

In addition, the products formed by lipoxygenases are converted by a divinyl ether synthase, a reductase, a peroxygenase and a hydroperoxide lyase (Blée, E. (1998) Prog. Lipid Res. 37, 33-72). Owing to these reaction opportunities and the non-specific ketol binding via AOS, AOC can be regarded as the first enzyme that makes jasmonate synthesis highly specific.

To date, a number of forms of lipoxygenases, allene oxide synthases and OPDA reductases have been cloned from plants, biochemically characterised and investigated in terms of their physiological importance (Rosahl, S. (1996) Z. Naturforsch. 51 c, 123-138; Song et al. (1993) Proc. Natl. Acad. Sci. USA 90, 8519-8523; Laudert, D. and Weiler, E. W. (1998) Plant J. 15, 675-684).

The object of the present invention was to provide nucleic acids and fragments thereof with which a procedure for the highly specific production of the aforementioned stereoisomer cis-(+)-OPDA from the JA biosynthesis pathway is made possible.

According to the invention, this task is solved by a nucleic acid that codes for a protein with the activity of the allene oxide cyclase and comprises a sequence selected from the following sequences:

a) nucleic acid that is obtainable by screening a plant gene bank with a probe and selection for allene oxide cyclase-positive clones;

b) nucleic acid that codes for a protein with the sequence selected from SEQ-ID nos. 2, 4, 6, 8, 10, 12, 14, 16;

c) nucleic acid that hybridises with a nucleic acid according to b);

d) nucleic acid which, taking account of degeneration of the genetic code, would hybridise with a nucleic acid according to b);

e) derivatives of a nucleic acid according to a)-d) that are obtained by substitution, addition, inversion and/or deletion of one or more bases;

f) nucleic acid with at least 53%, preferably at least 70% identity to the coding range of the nucleic acid according to SEQ ID no. 1;

g) complementary nucleic acid to a nucleic acid according to one of groups a)-f).

Using the present AOC clones, the possibility now exists for the first time of performing intensive physiological studies on the JA biosynthesis pathway, but also for the first time of producing JA and its derivatives by biotechnological means. The limiting step in specific jasmonate synthesis had up to now been the lack of specific producibility of the stereoisomer (9S)(13S)(cis(+)) of 12-oxo-phytodienic acid.

“Allene oxide cyclase” means an enzyme that catalyses cis(+) OPDA synthesis from the substrate 12,13-EOT, as shown in FIG. 1.

“Probe” means a nucleic acid that is suitable for scanning gene banks. However, the term is also understood to means agents, preferably proteins, that can demonstrate the presence of the expression product allene oxide cyclase. These include, among others, anti-allene oxide yclase antibodies.

Determination of the conditions for specific “hybridisation” between specified nucleic acids is within the limits of what is technically possible. Hybridisation with one of the sequences for a protein with the sequence selected from SEQ ID nos. 2, 4, 6, 8, 10, 12, 14, 16 preferred, preferably the sequence according to SEQ ID no. 1 under the following conditions: hybridisation with the sequence according to SEQ ID no. 1 with 2×SSC, 0.1% SDS at 50° C. is preferred, preferably for at least 30 minutes. Somewhat more stringent, likewise preferred hybridisation takes place with 1×SSC, 0.1% SDS at 50° C. Particularly preferred is hybridisation in which Express Hyb™ hybridisation solution manufactured by Clontech (USA), with hybridisation being performed at 60° C. for 18 hours and then washing being carried out with 2×SSC, 0.1% SDS at 50° C. for 30 minutes followed by 1×SSC, 0.1% SDS at 50° C. for 30 minutes.

According to the invention, the expression “% identical” refers to identity at nucleic acid level, which can be determined by known procedures, e.g. computer-supported sequence comparisons (Basic local alignment search tool, S. F. Altschul et al., J. Mol. Biol. 215 (1990), 403-410).

The expression “% identity” familiar to the expert designates the degree of relatedness between two or more nucleic acid molecules, which is determined by the match between the sequences. The “identity” percentage arises from the percentage of identical areas in two or more sequences, taking account of gaps and other sequence features.

The mutual identity of related nucleic acid molecules can be determined by known procedures. As a rule, special computer programs with algorithms that take account of the special requirements are used. Preferred procedures for determining identity initially produce the greatest match between the sequences investigated. Computer programs for determining identity between two sequences include, but are not restricted to, the GCG programme package, including GAP (Devereux, J., et al., Nucleic Acids Research 12(12):387 (1984); Genetics Computer Group University of Wisconsin, Madison, (Wis.)); BLASTP, BLASTN and FASTA (Altschul, S. et al., J. Molec Biol 215:403/410 (1990)). The BLAST X program can be obtained from the National Centre for Biotechnology Information (NCBI) and from other sources (BLAST Manual, Altschul S., et al., NCB NLM NIH Bethesda Md. 20894; Altschul, S., et al., J. Mol. 215:403/410 (1990)). The well-known Smith Waterman algorithm can also be used for determining identity.

Preferred parameters for the nucleic acid sequence comparison include the following:

| Algorithm: | Needleman and Wunsch, |
|---|---|
| | J. Mol. Biol 48: 443-453 (1970) |
| Comparison matrix: | Matches = +10, |
| | Mismatch = 0 |
| Gap Penalty: | 50 |
| Gap Length Penalty: | 3 |

The GAP program is also suitable for use with the above parameters. The above parameters are the default parameters for nucleic acid sequence comparisons.

Other exemplary algorithms, gap opening penalties, gap extension penalties, comparison matrices including those specified in the program manual, Wisconsin package, Version 9, September 1997, can be used. The selection will depend on the comparison to be performed and also on whether the comparison is performed between sequence pairs, with GAP or best fit being preferred, or between a sequence and an extensive sequence database, with FASTA or BLAST being preferred.

In a preferred embodiment, the nucleic acid according to the invention codes a protein with the sequence as indicated in the sequences with SEQ ID no. 2, 4, 6, 8, 10, 12, 14, 16, preferably in SEQ ID no. 2. Protein variants from the specific sequence can be produced using conventional methods via changes in the nucleic acid sequence (as by substitution, addition, inversion or deletion of one or more bases). Whether a new protein variant still exhibits the desired allene oxide cyclase activity can be readily demonstrated with the aid of appropriate enzyme tests, as specified in more detail below.

In another preferred embodiment, the nucleic acid is a cDNA, but genomic DNA is also suitable as an allene oxide cyclase-coding nucleic acid.

The following table provides a list of clones isolated with the DNA according to the invention in accordance with SEQ ID no. 1 of the method according to the invention.

| | lenght cDNA/ | homology to | | signal | | | |
|---|---|---|---|---|---|---|---|
| | number amino- | tomato AOC: | coding | sequence | localization | localization | |
| | acids | DNA/Protein | region | (putative) | (chromosome) | (cell) | SEQ ID No. |
| NtAOC1 | 1018 bp / 245 AS | 77,767% / | Nukleotid | Nukleotid | | Chloroplasten | 15 |
| | | 87,190% | 50-784 | 50-232 | | stromata | |
| AtAOC1 | 991 bp / 254 AS | 53,789% / | 69-830 | 69-302 | Chromosom 3 | Chloroplasten | 3 |
| | | 54,357% | | | | stromata | |
| AtAOC2 | 845 bp / 253 AS | 56,950% / | 49-807 | 49-279 | Chromosom 3 | Chloroplasten | 5 |
| | | 56,540% | | | | stromata | |
| AtAOC3 | 915 bp / 257 AS | 56856% / | 73-843 | 73-240 | Chromosom 3 | Chloroplasten | 7 |
| | | 55,967% | | | | stromata | |
| AtAOC4 | 820 bp / 254 AS | 58488% / | 24-785 | 24-185 | Chromosom 1 | Chloroplasten | 9 |
| | | 55,833% | | | | stromata | |
| MtAOC1 | 956 bp / 252 AS | 56,663% / | 20-775 | 20-187 | | Chloroplasten | 13 |
| | | 60,082% | | | | stromata | |
| HvAOC1 | 920 bp / 238 AS | 54,362% / | 62-775 | 62-202 | Chromosom 3 | Chloroplasten | 11 |
| | | 59,244% | | | | stromata | |

The fragments according to the invention of the nucleic acids used are characterised in that they can hybridise specifically with the aforementioned nucleic acid. “Specific hybridisation” means in this context that the expert can choose the hybridisation conditions in such a way that the fragment following hybridisation only produces a signal with the aforementioned nucleic acid and does not produce a signal with other nucleic acids also present in the sample. Such fragments can also be used for specific amplification of the nucleic acid according to the invention by means of PCR.

In another preferred embodiment, expression of the allene oxide cyclase in a host cell can be inhibited with the aid of the fragment, with this being made possible, for example, by the fragment in antisense orientation to a promoter. Inhibition of expression of the allene oxide cyclase by a fragment in sense orientation is, however, also possible. The “construct” containing a nucleic acid according to the invention is a combination of the aforementioned nucleic acid according to the invention and another nucleic acid with which the nucleic acid according to the invention is of course not linked. Thus, the construct can comprise AOC-coding sequences together with, for example, regulator sequences, vector sequences or sequences of fusion partners. In particularly preferred constructs, the sequence according to the invention is present in a plasmid.

The host cells according to the invention containing the nucleic acid according to the invention are characterised in that they contain the nucleic acid in a quantity/number of copies that is not thus present in the host type. Alternatively, the host cells may also be cells whose wild type does not contain the nucleic acid according to the invention but exhibit this following incorporation of the nucleic acid.

The nucleic acids according to the invention can be introduced with conventional transformation techniques in any cells, including bacteria, and also plant cells, animal cells, including insect cells, and human cells.

Particularly preferred is the introduction of the nucleic acid according to the invention in plant cells or, for example, plant tissue or plant organs.

In a preferred embodiment, the host cell or the cell unit contains the nucleic acid integrated in the genome. As a rule, the integration site will not be the location in which the wild type contains the allene oxide cyclase gene, where present.

The incorporation of the sequences according to the invention under control of suitable elements that control the expression of nucleic acid, which are familiar to the expert, can be described AOC [sic] in a large quantity and with great purity. The techniques for the expression and isolation of protein from the host cells are familiar to the expert.

In another preferred embodiment, the protein is a fusion protein comprising allene oxide cyclase or a fragment thereof and a non-allene oxide cyclase protein sequence. Preferred fusion partners are sequences from allene oxide synthase and reductase, as are to be found in the jasmonate biosynthesis pathway. Particularly preferred is full allene oxide cyclase in connection with full allene oxide synthase or OPDA reductase.

The production of antibodies, which are oriented against the protein according to the invention, is familiar to the expert. The extraction of antisera and the production of polyclonal antibodies following immunisation of a host organism with the protein according to the invention are considered for this. Alternatively, however, hybridoma technology can also be used to produce monoclonal antibodies.

With the aid of the nucleic acids according to the invention, new transgenic plants and plant parts can now be produced for the first time by the nucleic acid according to the invention being introduced in a starting plant cell and the more complex plant tissue right through to the entire plant being regenerated from the initially transformed plant cell. The techniques needed for this are available to the expert.

Allene oxide cyclase activity in a plant or its parts can also be influenced via the nucleic acid according to the invention. Allene oxide cyclase activity in the plant or in the plant cells can be increased via the introduction of the nucleic acid, for example with a strong promoter. This effect can also be achieved by increasing the number of copies of the allene oxide cyclase-coding sequences. On the other hand, the endogenous expression of the allene oxide cyclase gene can be reduced or even completely blocked by the introduction of the nucleic acid according to the invention, for example in antisense orientation to a promoter. This then leads to a reduction in allene oxide cyclase activity in the corresponding plant part.

Lastly, the procedure according to the invention allows for the first time for the highly selective production of the metabolite 9,13-cis-(+)-12-oxophytodienic acid in a large quantity. This metabolite is the essential and decisive starting stage for the production of jasmonic acid in its naturally occurring enantiomeric form in large quantities. The said metabolite had previously been virtually unavailable as there was no possibility of selectively producing cis-(+)-OPDA from the precursor molecule, 12,13-EOT. Only mixtures of various metabolites were available. These metabolite mixtures in turn prevented the synthesis of jasmonate in large quantities and with a high degree of purity.

With the AOC-coding gene becoming available, all means are now at the expert's disposal for producing jasmonic acid in large quantities and with a high degree of purity from, for example, α-linolenic acid. The full biosynthesis pathway can now be completely established in a host organism via recombinant DNA techniques. A decisive step that now allows for the biotechnological production of jasmonic acid in large quantities was the provision of the nucleic acid according to the invention. The latter has made it possible for the first time to make available cis-(+)-OPDA with a high degree of purity and in large quantities as starting material for further jasmonic acid biosynthesis.

The nucleic acid according to the invention also allows for the production of new transgenic crops with modified characteristics. The new characteristics include an increase in the pathogenic defence or pathogenic resistance of plants. As a result of the increased expression of AOC following the introduction of the nucleic acid according to the invention in the plant material, an increase in metabolites involved in pathogen defence occurs. Increased preparedness for defence against herbivores can also be brought about in the same way. This effect is based on increased induction of the wound response following introduction and expression of the nucleic acid according to the invention in the plant material. The nucleic acids according to the invention also allow for optimisation of the plant-useful insect-pest interaction. This is based on the expression of the nucleic acid according to the invention in the plant leading to a modified smell emission spectrum in the plant, with insects that help to eliminate insects that are harmful to plants (tritrophic interaction) particularly being attracted. Furthermore, biomass formation by the modified plants can be increased by introduction of the nucleic acid according to the invention in plant material. This is based on optimisation of the mycorrhizing capacity of roots as a response to expression of the nucleic acid according to the invention.

Furthermore, the carbohydrate balance of the plants can be modified via the nucleic acid according to the invention. This is based on enzymes involved in assimilate partitioning (e.g. invertases) being regulated by jasmonate. A modified jasmonate level thus modifies the activity of the said enzymes. An assimilate shift can in this way be achieved, with increased accumulation in storage organs and in seed ripening being possible.

Lastly, the nitrogen balance can be modified via the nucleic acid according to the invention, particularly if the nucleic acid according to the invention is expressed in the vegetative storage organs. Vegetative storage proteins, as present for example in soya beans, represent a form of N and protein storage. Expression of these proteins is inducible by wounds and jasmonate. Accordingly, an increased AOC expression with increased jasmonate formation results in an increase in the protein content in vegetative storage organs.

Furthermore, the UV protection of plants can be optimised with the aid of the nucleic acid according to the invention. Anthocyan formation is also inducible by jasmonate. A targeted increase in the endogenous jasmonate level, for instance in epidermal cells as a result of the increased AOC expression, may lead to an increase in the anthocyan quantity in these cells. Anthocyans are in turn known as UV protectants. Thus, plants with an increased anthocyan content in epidermal tissue are less sensitive to UV stress.

Furthermore, the generation of male sterility by the introduction of nucleic acid according to the invention in pollen-forming tissue of the flowers is possible. The nucleic acid is in this context preferably used in antisense orientation under a regulator element, as a result of which the internal expression of AOC is reduced. The tissue-specific expression of the nucleic acid introduced can also be achieved by means of tissue-specific promoters.

Lastly, developmental processes in plants, particularly flower development, can also be modified via the nucleic acid according to the invention. In flower development, α-linolenic acid plays an important role. α-linolenic acid is the precursor of jasmonic acid in the tapetum, the pollen-forming tissue of the flowers, and represents the only unsaturated fatty acid there. Mutants with disturbances in α-linolenic acid formation are jasmonate-deficient. These mutants also exhibit male sterility, like the jasmonate-insensitive mutants. It follows from this that lines displaying male sterility can be produced by inhibition of jasmonate formation via a reduction in AOC expression. As flowers, for example tomato flowers, display a high expression of specific invertases and also a high jasmonate content, the characteristics of the flowers can be influenced via a modified jasmonate content.

FIG. 1 shows a diagram of jasmonate biosynthesis

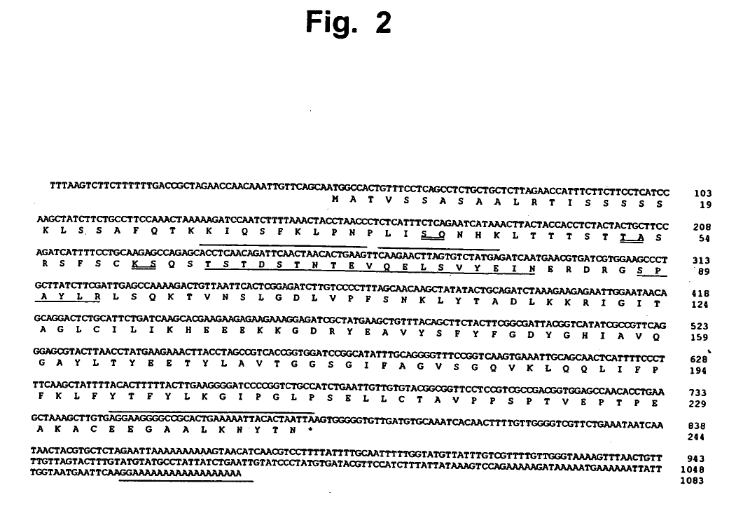

FIG. 2 shows the nucleotide sequence of the allene oxide cyclase cDNA clone from tomatoes and the amino acid sequence derived therefrom. Arrows printed in bold indicate the sequences used for the RT-PCR. Broken arrows indicate the sequences used to amplify a fragment that codes for a truncated version of the allene oxide cyclase, used in an overexpression assay. Putative restriction interfaces for chloroplastid signal peptides that have been predicted using the computer program ChloroP V1.1 are double-underlined.

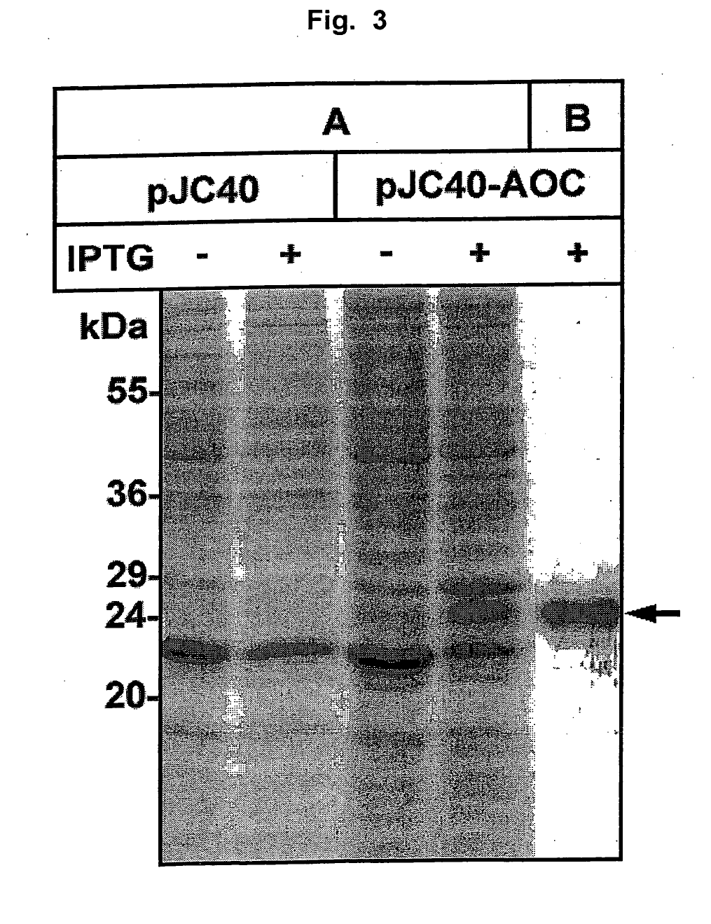

FIG. 3 shows the overexpression of a truncated tomato AOC protein in bacteria whose translation starts at the amino acid radical in position 64. The corresponding partial cDNA was subcloned in the vector pJC20 and cloned for purification of the protein in a vector pJC40 bearing a histidine tail. The E. coli strain BL21 DE3(pLysS) was used for transfection. Both extracts of bacteria that had been transformed with the vector pJC40 alone and extracts of bacteria that had been transformed with partial AOC-cDNA (in vector pJC40), either in the absence (−) or in the presence of IPTG (+), were attracted and then separated by gel electrophoresis via SDS-PAGE. The gel was stained with Coomassie Blue (A) or a corresponding blot was sampled with an anti-AOC antibody (B). An arrow indicates the AOC protein.

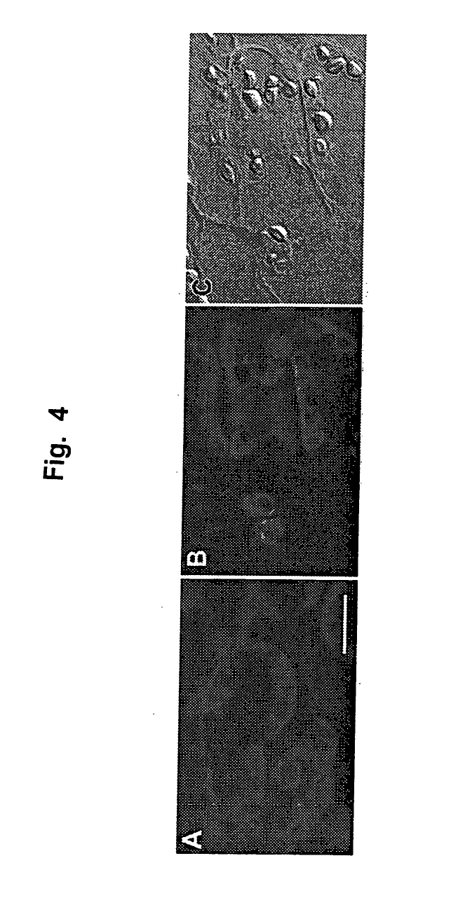

FIG. 4 shows the location of the AOC protein in tomato leaves following immunocytochemical identification. Leaf cross-sections incubated for 24 hours in 50 μmol JAME were treated either with pre-immune serum (A) or with anti-AOC antibodies oriented against the purified recombinant AOC protein from tomato (B), followed by a second treatment with a second antibody, which was BODIPY-labelled. Unlike the yellow-brown autofluorescence following treatment with the pre-immune serum, a clear fluorescence marking in the chloroplasts in B showed the presence of the AOC protein. Starch grains in the chloroplasts that were visible in B as non-fluorescing areas were made visible via differential interference contract©. The scale is quoted in μm.

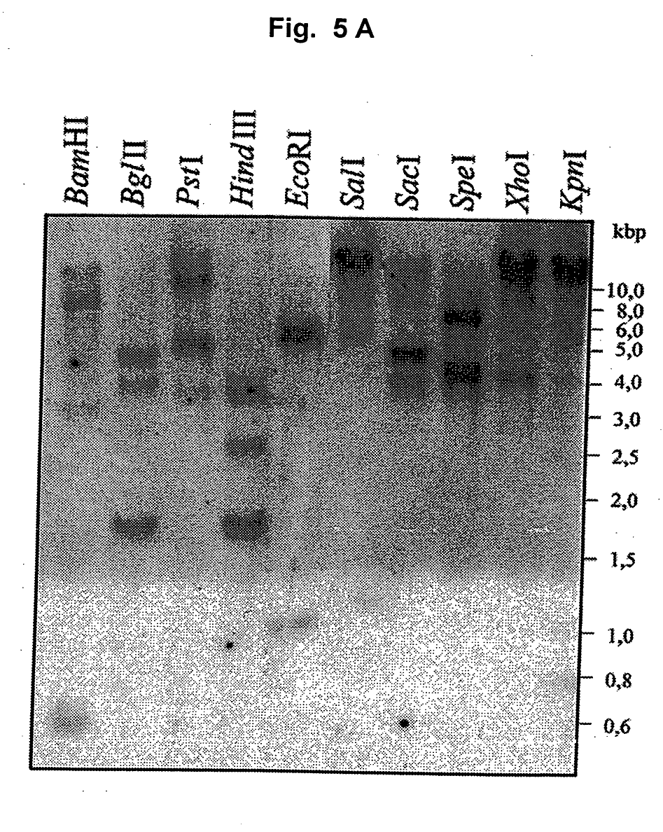

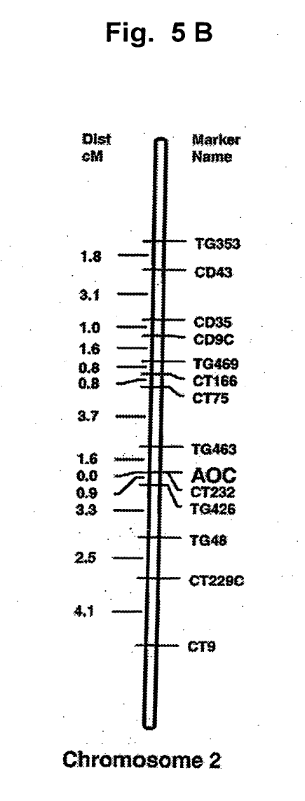

FIG. 5 shows the southern blot analysis of genomic DNA from tomato (A) and the RFLP analysis of the AOC locus (B). The cleavage with the various restriction enzymes showed that the AOC gene is present as a single copy.

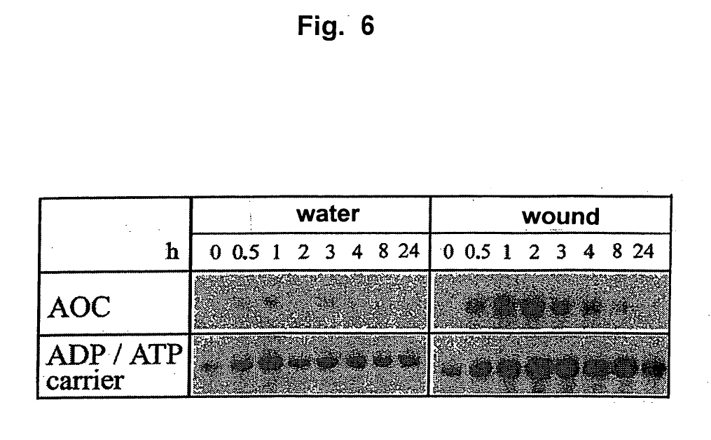

FIG. 6 shows the northern blot analysis of the accumulation of AOC-mRNA in wounded tomato leaves. Per trace, 10 μg total RNA were loaded. They originated from unwounded (water) or wounded leaves, that were harvested at different times after the start of the experiment.

The following examples elucidate the invention.

Genomic Analysis of the AOC Genes

For the southern blot analysis, genomic DNA from tomato was digested with the restriction enzymes BamHI, BgIII, PsfI, HindIII, EcoRI, EcoRV and EcoRII (FIG. 5A). Following cleavage with the first two enzymes, three bands were detected. As these enzymes exhibited two cleavage sites in the insert, it can be assumed that the AOC in the tomato genome is present as so-called “single copy” gene. This assumption is further supported by the band pattern, which was obtained by restriction with the other enzymes. In the case of cleavage with PsfI and HindIII, two bands were detected, with these enzymes each exhibiting cleavage sites once in the cDNA insert, while only one band was detected with the last three enzymes that exhibited no restriction cleavage sites in the cDNA insert. The results of the cleavages are set out in FIG. 5A. They correspond to the mapping data according to which a gene location on chromosome 2 was detected for AOC (FIG. 5B).

Physiology of AOC Expression

It was shown many times that the accumulation of JA forms an important part of the signal transduction chain in the wound response. In the tomato, chloroplastid lipoxygenase is up-regulated if the plant is wounded, while Arabidopsis plants that are inhibited by cosuppression with a specific chloroplastid lipoxygenase showed no increased in JA concentrations as a wound response following wounding. In addition, the strict spatial and temporal regulation of the second enzyme of the biosynthesis pathway, AOS, emphasises the importance of the activation of corresponding enzymes of JA biosynthesis in the accumulation of JA during wound response in Arabidopsis. As AOC is the enzyme that catalyses the stage in the biosynthesis of JA in which the “correct”, naturally occurring stereoisomeric form is formed, it was interesting to know whether the expression of AOC contributed to an increase in JA concentrations following wounding. As shown in FIG. 6, the level of AOC mRNA in tomato leaves rises 30 minutes after wounding of the tomato leaves. Maximum induction was observable after two hours, and the control quantity was achieved again after 8 hours. This finding correlates closely with the JA quantities measured after wounding and that also exhibited a transient accumulation with an increase after 1 hour. This finding demonstrates an important physiological function of AOC in the regulation of JA concentrations during the wound response in tomato plants.

Another extremely important function of AOC seems to lie in the influence on the development of floral organs. A high degree of expression was detected in pistil tissue, mature floral petals, red fruit and, to a lesser extent, in stamens. On the other hand, no expression was detected in young, developing buds. Mutants that are defective in the JA signal transduction chain, such as Arabidopsis coi1 or fad3-2 fad7-2 fad8 mutants, exhibit male sterility. This shows the importance of JA in floral development. It was also shown that AOS genes (the synthase genes) are strongly expressed in floral organs of Arabidopsis thaliana, which suggests that JA is possibly synthesised in flowers. The tissue-specific expression of the AOC gene in vascular bundles, and, all the more surprisingly, in ovules of young tomato flowers was also shown by immunocytochemical analysis. The role of AOC and JA in flower development is based essentially on the strict correlation of AOC expression and the rise in JA levels. The other enzymes involved in JA biosynthesis were also identified by the findings of Vick and Zimmermann and of Hamberg (Vick, B. A. & Zimmermann, D. C. (1983) Biochem. Biophys. Res. Commun. 111, 470-477; Hamberg, M. (1988) Biochem. Biophys. Res. Commun. 156, 543-550). Over the last ten years, the characterisation and cloning of these enzymes have made good progress, and clones of LOX (Rosahl, S. (1996) Z. Naturforsch. 51.c, 123-138), AOS (Song, W. C. & Brash, A. R. (1991) Science 253, 781-784; Laudert, D., Pfannschmidt, U., Lottspeich, F., Holländer-Czytko, H. & Weiler, E W. (1996) Plant Mol. Biol. 31, 323-335) and OPDA reductases (Schaller, F. & Weiler, E. W. (1997) J. Biol. Chem. 272: 28066-28072; Biesgen, C. & Weiler, E. W. (1999) Planta 208: 155-163) are now available. With the isolation of a cDNA clone coding for AOC that is described in the present invention, all enzymes that lead to the first physiologically active cyclopentenone, OPDA, are now cloned. In addition, this enzyme seems to be of particular importance as it decisively determines the stereochemistry of the cyclopentanones and plays a crucial role in the control of one of the possible oxylipin biosynthesis pathways that lead to the biosynthesis of jasmonates. Using this AOC clone and the other enzymes, intensive physiological studies are now possible, together with biotechnological applications with regard to the jasmonate biosynthesis pathway.

Plant Material

To identify genes that code for AOC, different material was used. Plant material from barley (Hordeum vulgare L.cv. Salome) and tomato (Lycopersicon esculentum Mil. cv. Moneymaker) was raised in soil under greenhouse conditions with 16 hours of light (with a minimum intensity of 130 μmol/m²/s) at 25° C. Primary leaves of the barley plants were harvested 7 days after germination, cut into 5 cm long segments, measured from 1 cm below the leaf tip. These leaf fragments were incubated in Petri dishes in a 1 mol sorbitol solution. Tomato plants were cultivated for 8 weeks, the secondary lead was cut off, wounded with a standard commercial toothed wheel and then incubated in distilled water under continuous white light (at an intensity of 120 μm/m²/s).

Analysis of Endogenous JA and OPDA Quantities

In order to determine quantitatively the concentration of non-esterified 12-oxo-PDA and liholenic acid and the steric analysis of 12-oxo-PDA, unwounded leaves of tomato plants (15 to 20 g) or leaves (3-5 g) that had been previously wounded were used as starting material. The tissue was quick-frozen in liquid nitrogen and then extracted with ethanol. Deuterium-labelled 12-oxo-PDA ([²H₅]-12-oxo-PDA) and deuterium-labelled linolenic acid ([²H₅]-linolenic acid) were added to small portions of the extract. The quantities of 12-oxo-PDA and linolenic acid were then determined via mass spectrometry methods. Remaining portions of the extract to which the labelled substances had not been added were subjected to solid-phase extraction and separated via RP-HPLC. The 12-oxo-PDA thus isolated was subjected to steric analysis. The quantities of JA were determined by the method as described in Kramell et al. FEBS Lett. 414, p. 197, (1997).

Enzyme Assay

AOS activity was determined in 50 mM potassium phosphate with pH 6.7 in a total volume of 1 ml. The reaction was initiated by the addition of the fatty acid hydroperoxide, and the decrease in absorption was measured at 235 nm. To determine the kinetic parameters of AOS from maize, the initial reaction speed was determined for 14 concentrations in the range 5-90 μm for every substrate. The Kₘ and v_{Max} values were directly calculated with the aid of the Michaelis-Menten equation and plotted in accordance with the Eadie-Hofstee diagram.

Analysis of the Cyclisation Products of the Allene Oxides

Fatty acid hydroperoxides (150 μm) were agitated at 0° C. for 10 minutes with 0.5 ml of a suspension from maize seed membranes (0.5 mg protein; 28 ncat using 13 (S)-HPOT as substrate) in 0.1 m potassium phosphate buffer pH 6.7 and 2.5 ml of the AOC preparation in ammonium sulphate/20 mm tris-HCL buffer pH 7.5 (total assay volume: 5 ml). The activities of AOC from maize and potatoes that were added in this assay were equivalent to 178 nmol PDA or 156 nmol PDA, as measured by the method based on RP-Radio HPLC. Controls in which the AOC solution was replaced by potassium phosphate buffer were performed in parallel. The incubations were ended by the addition of 10 ml ethanol and the reaction products were extracted with diethyl ether, methylated and subjected to TLC (with the solvent system: ethyl acetate/toluene 15:18 vol/vol.). The bands were made visible under UV light following spraying with 26-dichlorofluorescein, and the R_{f} value of the cyclopentenone bands (of the methyl esters of 12-Oxo-PDA and its homologues and analogues) was recorded. A broad zone of the silicate gel containing bands based on the methyl ester of cyclopentenone, hydroxy fatty acid and α-ketol was scratched off and diluted with ethyl acetate. An aliquot of the material was trimethylsilylated and subjected to analysis via GLC and GC-MS. The remaining portion of the material was used for steric analyses of the cyclopentenones, (cf. Hamberg, M. and Fahistadius, P. in Arch. Biochem. Biophys. 276, pp. 518-526, 1990 and Hamberg, M. et al., in Lipids 23, pp. 521-524, 1988).

Overexpression of the Allene Oxide Cyclase

Both the entire coding region including the start codon and a 5′-deleted version of the clone, starting with nucleotide 235 (FIG. 2), were amplified via PCR, provided with 5′NdeI- and 3′EcoRI restriction interfaces respectively and cloned in the expression vectors pJC20 and pJC40 respectively (Clos and Brandau Protein. Expr. Purif. 5, p. 133, 1994). The plasmids obtained were transformed in the bacterial strain BL21 DE3(pLysS). Cultivation of the bacteria took place in LB medium up to a value of 0.5, measured for an OD of 600 nm. The bacterial suspension was induced for 4 hours by 1 mM IPTG, then centrifuged, subjected to two freezing and thawing cycles and then lysed by ultrasound in 20 mM trisHCl pH 7.5, 0.5 M NaCl, 0.1% Tween 20 and 10% glycerol. After further centrifugation, the supernatants were further used for measurement of AOC activity. Enzyme activity was on the one hand measured via a radioactive assay in accordance with the method, as specified in more detail below, and by determination of the enantiomeric composition of OPDA. The hybridisation conditions were as follows: using Express Hyb™ hybridisation solution manufactured by Clontech (USA), hybridisation was carried out at 60° C. for 18 h and then washing was performed with 2×SSC, 0.1% SDS at 50° C. for 30 minutes followed by 1×SSC, 0.1% SDS at 50° C. for 30 minutes. The recombinant protein expressed by the expression vector pJC40 was purified via affinity chromatography on a Ni-nitrilotriacetic acid agarose (NiNTA, Qiagen). The purity of the protein was controlled via SDS-PAGE gel electrophoresis. The purified recombinant AOC enzyme was then used to produce a polyclonal rabbit antibody.

Northern and Southern Blot Analysis

The northern blot analysis was performed in accordance with Maniatis et al. Molecular Cloning: A Laboratory Manual, Cold Spring Harbour (1989) with 10 μg total RNA per trace. The blots obtained were hybridised at 65° C. for 16 hours with a radioactively labelled sample of the tomato AOC-cDNA clone, consisting of the full-length cDNA sequence (see FIG. 2). Hybridisation with the sequence in accordance with SEQ ID no. 1 with 2×SSC, 0.1% SDS at 50° C., preferably for at least 30 minutes, is preferred. A somewhat more stringent, likewise preferred hybridisation takes place with 1×SSC, 0.1% SDS at 50° C. Particularly preferred is hybridisation in which Express Hyb™ hybridisation solution manufactured by Clontech (USA) is used, with hybridisation being carried out at 60° C. for 18 hours and then washing being performed with 2×SSC, 0.1% SDS at 50° C. for 18 hours followed by 1×SSC, 0.1% SDS at 50° C. for 30 minutes. Uniform gel loading of the samples was controlled via ethidium bromide staining of the ribosomal RNA.

For the southern blot analysis, 10 μg genomic DNA with the indicated restriction enzymes were digested and separated via gel electrophoresis. Following vacuum transfer to a nylon membrane, hybridisation took place as described above for the northern blot.

Immunocytochemistry

Small pieces of young leaves were fixed in accordance with Huse et al (1996) Plant Cell Physiol. 37 641-649, packed in PEG and cut. Sections that were 2 μm thick were treated with a rabbit anti-AOC antibody (in a dilution of 1:2000). Further incubation was performed with a goat anti-rabbit IgG antibody, conjugated with BODIPY (Molecular Probes, Eugene, Oreg.). Following immune labelling, the sections were incubated in Citifluor/glycerol. Control experiments were conducted with pre-immune serum. Fluorescence of the immuno-labelled AOC enzyme was made visible with a Zeiss “Axioscope” epifluorescence microscope using the corresponding filter combination.

Cloning of AOC

For RNA isolation purposes, tissue was used in which the endogenous JA concentration accumulates on the basis of induction. Thus, sorbitol-stressed leaves from barley (Lehmann et al., Planta 197 p. 156, 1995) and wounded tomato leaves (Peña-Cortes et al., Proc. Natl. Acad. Sci USA, p. 4106, 1995) were used as starting material for further RNA isolation. Whereas no specific PCR products were obtained for barley,.a weak band of around 900 bp fragment size was amplified following RT-PCR with RNA from wounded tomato leaves. This PCR product was used as a probe for the screening of the tomato cDNA bank, which led to isolation of a 1 kb large cDNA clone. This size roughly corresponded to the signal magnitude that was detected in the northern blot analyses. It was concluded from this that the isolation involved a full-length clone. The first start codon is located at position 47, and this is preceded by a stop codon in position 16. The protein-coding region comprises 732 bp that code for a protein of 244 amino acids with a calculated molecular weight of 26 kDa (FIG. 2). The deviation of around 4 kDa for the derived molecular weight of protein from tomato was determined by SDS-PAGE gel electrophoresis and might, at least in part, be the result of the post-translational removal of amino acids at the N-terminus of the tomato protein.

Overexpression of AOC

To identify the protein described in the present invention, coded by the nucleic acid described, as an AOC protein, overexpression experiments were performed to measure AOC activity. First of all, the entire coding region without the start codon for methionine in the expression vector pJC20 was cloned and introduced in the vector pJC40 bearing a histidine tail for further purification. The absent methionine was supplemented via a 5′NdeI restriction interface. Following induction via IPTG, only weak expression of the recombinant protein on a SDS-PAGE gel was observed. However, following column chromatographic purification (NiNTA) of the protein bearing a histidine tail, a band of around 26 kDa was detected. However, neither the raw extract of the bacteria nor the purified protein exhibited AOC activity. The lack of enzymatic activity in the full-length clone from tomato might be a result of the false post-translational modification in the bacteria. To investigate this possibility further, a fragment of the tomato sequence was amplified, which codes for a truncated protein that begins on amino acid radical 64. Here, too, the start codon was supplemented via a NdeI restriction interface. Following corresponding induction of the bacteria, SDS-PAGE gel electrophoresis was performed and yielded a clear band at 22 kDa which was not detected in the control bacteria that had been transformed only with an empty vector (FIG. 3). The same band was also detected in non-induced bacteria, which might be due to inadequate repression of the bacterial expression system in the absence of induction molecules. All the bacterial extracts were then analysed for AOC activity. As shown in Table 1, see below, no activity was detected in the control bacteria, while a high level of specific activity of around 15000 nMol OPDA/mg protein was detected in the induced transformed bacteria. As expected for the protein sample of SDS-PAGE gel, the non-induced bacteria also showed AOC activity; however, this was about 10 times lower than for the induced bacteria. AOC activity of the recombinant protein was sensitive to proteinase K digestion and was able to be inhibited by 12,13-epoxyoctadecenoic acid, a specific AOC inhibitor, which further supports the specificity of the recombinant protein. A specific property of the AOC reaction is the competing reaction between chemical cleavage of the unstable allene oxide substrate, resulting in a racemic mixture of OPDA, and the enzymatic transformation to enantiomeric OPDA. As a result, a protein can ultimately only be identified as AOC if the specific enantiomer 9(S), 13(S)-OPDA was formed and detected.

Steric analysis of the reaction products- revealed that the recombinant protein had formed virtually exclusively the said 9(S),13(S) enantiomer of OPDA. The performance of a proteinase-K digestion and the addition of 12,13-epoxyoctadecenoic acid reduced the concentration of this OPDA enantiomer to a concentration, which was also observed following chemical cleavage of the unstable allene oxide. All in all, the results of the AOC activity measurements showed that the isolated clone from tomato codes for an AOC. Interestingly, the specific activity of the N-terminal protein bearing a histidine tail was about 20 times less than that of a similarly analysed protein without a histidine tail. Together with the observation that only the truncated protein showed a high level of activity, it seems likely that additional amino acids in the N-terminus might possibly disturb formation of the protein dimer.

Intracellular Localisation of AOC

It has been reported that most enzymes of the oxylipin biosynthesis pathway are localised in the chloroplast. An N-terminal amino acid sequence analysis of the cloned LOXs from barley (Vörös et al., Eur. J. Biochem. 251, p. 36, 1998), Arabidopsis (Bell et al., Proc. Natl. Acad. Sci USA, p. 8675, 1995) and tomato (Heitz et al., Plant Physiol 114, p. 1085, 1997) and also partially performed import studies in chloroplasts showed both the presence and the function of putative chloroplastid signal peptides. The AOS from flax (Song et al., Proc. Natl. Acad. Sei 90, p. 8519, 1993) and Arabidopsis (Laudert et al., Plant. Mol. Biol., 31, p. 323, 1996) also exhibited possible chloroplastid signal peptides and, like the AOS from barley, were purified together with chloroplasts. Enzyme activities of LOX, hydroperoxide lyase and AOS were detected by biochemical data in the outer coat membrane of chloroplasts (Blee and Joyard, Plant Physiol. 110, p. 445, 1996). In the case of AOS and LOX, their chloroplastid location was also confirmed immunocytochemically. Investigation of the N-terminal region of the AOC from tomato also yielded additional structural data for a chloroplastid signal peptide. The latter is rich in serine residues (26% in the first 50 amino acids), the start codon for methionine is followed by an alanine residue, and the first ten amino acids do not have a charged amino acid residue. Computer analysis of the first 100 amino acids using the Chlorop V1.1 program yielded a possible chloroplastid location for the protein with a putative cleavage site between position 93 and 94. However, this putative cleavage site is highly unlikely on various grounds. Other derived possible cleavage sites might represent amino acid residues in positions 41, 53 and 60. To determine the location of AOC experimentally, an immunocytological experiment with the antibody against the recombinant AOC was conducted. Cross-sections from tomato leaves were to this end incubated with this antibody and showed significant fluorescence in the chloroplasts (FIG. 4). The autofluorescence of the chloroplasts was shown by tissue cross-sections that were treated without the first antibodies. This confirms the data from the sequence analysis that indicated that AOC is a chioroplastid protein. Unlike AOS, which is located in the outer coat membrane of chloroplasts, AOC is a soluble protein. As the substrate is highly unstable, it seems reasonable to suppose that AOC is located in the vicinity of AOS, in order to ensure efficient substrate transformation to 9(S),13(S)-OPDA. In addition, neither ketols nor racemic OPDA have so far been detected in plants, which suggests that the chemical breakdown of allene oxide does not take place in vivo. To investigate this point further, quantities and the stereoconfiguration of endogenous 12-oxo-PDA in tomato leaves was determined. In a representative experiment with unwounded leaves, concentrations of the non-esterified 12-oxo-PDA and linolenic acid of 2 ng/g and 206 ng/g fresh weight respectively were determined. The steric analysis revealed that the quantities of OPDA were formed virtually completely (>99%) from the naturally occurring 9(S),13(S) enantiomer. The quantities of OPDA and linolenic acid rose to 187 ng/g (90 times) and 1813 ng/g (9 times) respectively within 30 and 180 minutes following mechanical tissue wounding. Steric analysis of wound-induced OPDA revealed exclusive formation (>99%) to the 9(S),13(S) stereoisomer. It might be concluded from this that AOS and AOC are either spatially located very close in the chloroplast or are even loosely associated in the chloroplast.

Determination of the Enzyme Activity of AOC

The reaction batch consisted of the 100,000 g pellets of the analysed tissue homogenate resuspended in 50 mM tris-HCl (pH 7.5) with an AOS activity of 7 ncat and the sample to be investigated. The volume was filled to 625 μl with 50 mM tris-HCl (pH 7.5) and the enzyme reaction was initiated by the addition of 2.6 μl 10 mM [1-¹⁴C]13(S)-HPOT (final concentration 41 μM; 0.83 kBq [0.022 μCi]). The batch was incubated on ice for 10 minutes. The reaction was stopped by the addition of 750 μl MeOH, the reaction mixture was acidified with 2 N HCl and the products extracted with 4 ml diethyl ether. The organic phase was evaporated and the residue absorbed in 110 μl acetonitrile/H₂O/HOAc (55/45/0.02 vol./vol./vol.). HPLC separation of the reaction products took place isocratically (acetonitrile/H₂O/HOAc 55/45/0.02 vol./vol./vol.) on a eurospher 100 C18 column (5 μm, 200×4.6 mm; Knauer Berlin) with a flow rate of 1 ml/min. Quantification of the products was performed by measurement of radioactivity via a flow cell filled with solid scintillator (yttrium silicate 0.4 ml, RSM 100 Radioactivity Monitor Analyser, Raytest Isotopenmeβgeräte GmbH, Strubenhardt). The quantity of OPDA was compared firstly with the peak area of the α-ketol and secondly with the sum of all peak areas appearing on the chromatogram and the AOC activity was calculated as follows:

$$[ \frac{( PDA {/}_{α - Ketol} ) - 0 , 17}{PDA {/}_{α - Ketol}} * PDA {/}_{total} ] * X$$

where “X” is the quantity of hydroperoxide (in mmol) used in the test and “total” represents the sum of all peaks appearing on the chromatogram. As a result of this equation, the quantity of OPDA formed enzymatically and thus the value for AOC activity with the unit [nmol OPDA] is obtained.

In order to determine the isomeric and enantiomeric composition of OPDA, the AOC enzyme test was conducted with non-radioactively labelled 13(S)-HPOT. To determine the cis/trans isomer ratio of the OPDA, the methylated reaction products were separated via GC on an SPB-1 column (30 m, 0.25 mm, coating thickness 0.25 μm; Supleco, Deisenhofen). The injection temperature was 100° C., which was increased to 175° C. at 15° C./minute. After 2 minutes at 175° C., the temperature was raised to 230° C. at 2.5° C./minute.

In order to determine the enantiomer ratio of OPDA, the extracted reaction products were first heated at 190° C. for 10 minutes and converted to the methyl esters by the addition of 1 ml ethereal diazomethane. Separation of-the methylation products was carried out on an HP 6890 GC system (Hewlett Packard) on a Megadex-5 GC column (12 m, 0.25 mm, coating thickness 0.25 μm) with a helium flow of 2 ml/min. The temperature for the injection was 150° C., which remained constant for the first 15 minutes and was then raised to 200° C. at 1° C./min.

TABLE 1
AOC activity and composition of OPDA,
as obtained via recombinant AOC

| | specific AOC activity | | |
|---|---|---|---|
| | nmol OPDA per mg | | % (S/S) of total |
| | protein | % | OPDA |
| pJC20/pJC40 | | 0 | 49 |
| pJC20 + Insert − IPTG | 1.202 | 7 | 55 |
| pJC20 + Insert + IPTG | 15.741 | 100 | 96 |
| +Proteinase K | 944 | 6 | 51 |
| +Inhibitor (100 μM) | 1.259 | 8 | 56 |
| pJC20 + Insert + IPTG | 651 | 4 | not determined |

1. Nucleic acid that codes for a protein with the activity of allene oxide cyclase from jasmonate biosynthesis, selected from
a) nucleic acid that is obtainable by screening a plant gene bank with a probe and selection for allene oxide cyclase-positive clones;
b) nucleic acid that codes for a protein with the sequence selected from the SEQ ID nos. 2, 4, 6, 8, 10, 12, 14, 16;
c) nucleic acid that hybridises with a nucleic acid according to b);
d) nucleic acid that would hybridise, taking account of the degeneration of the genetic code, with a nucleic acid according to b);
e) derivatives of a nucleic acid according to a) to d) that are obtained by substitution, addition, inversion and/or deletion of one or more bases;
f) nucleic acid with at least 53%, preferably at least 70% identity to the coding area of the nucleic acid according to SEQID no.;
g) complementary nucleic acid to a nucleic acid according to one of groups a) to f).

2. Nucleic acid according to claim 1, characterised in that it comprises the coding sequence according to one of the sequences selected from one of SEQ ID nos. 1, 3, 5, 7, 9, 11, 13, 15 or derivative derived therefrom by substitution, addition, inversion and/or deletion of one or more bases.

3. Nucleic acid according to one of claims 1 to 2, characterised in that it codes for a protein sequence selected from one of SEQ ID nos. 2, 4, 6, 8, 10, 12, 14, 16.

4. Nucleic acid according to one of claims 1 to 3, characterised in that it is a DNA, preferably a cDNA.

5. Fragment of a nucleic acid according to one of claims 1 to 4, characterised in that it comprises at least 10 nucleotides, preferably at least 50 nucleotides, and quite particularly preferably at least 200 nucleotides.

6. Fragment according to claim 5, characterised in that it can inhibit, in antisense orientation to a promoter, the expression of an allene oxide cyclase in a host cell.

7. Construct containing a nucleic acid according to one of claims 1 to 4 and/or a fragment according to one of claims 5 or 6 under the control of the expression-regulating elements, preferably of a promoter.

8. Construct according to claim 7, characterised in that the nucleic acid or the fragment is located in antisense orientation to the regulatory element.

9. Construct according to one of claims 7 or 8, characterised in that it is present as plasmid.

10. Host cell containing a nucleic acid according to one of claims 1 to 4 and/or a fragment according to one of claims 5 or 6 and/or a construct according to one of claims 7 to 9.

11. Host cell according to claim 10, characterised in that it is selected from bacteria, yeast cells, plant cells, animal cells and/or human cells.

12. Plant cell and/or plant tissue and/or plant organ and/or seed and/or plant containing a nucleic acid according to one of claims 1 to 4 and/or a fragment according to one of claims 5 or 6 and/or a construct according to one of claims 7 to 9.

13. Host cell and/or seed and/or plant cell and/or plant tissue and/or plant organ and/or transgenic plant according to one of claims 10 to 12, characterised in that the nucleic acid or the fragment or the construct is integrated in a position of the genome that does not correspond to its natural position in the wild type.

14. Protein obtainable by expression of a nucleic acid according to one of claims 1 to 4 in a host cell.

15. Fusion protein comprising the protein according to claim 14.

16. Fusion protein according to claim 15, with the fusion partner of the protein according to claim 15 being selected from the jasmonate biosynthesis pathway from:
a) allene oxide synthases and
b) reductases

17. Antibody that reacts with a protein according to one of claims 14-16.

18. Process for the production of a transgenic plant and/or plant cell and/or plant tissue and/or plant organ comprising the following steps:
introduction of a nucleic acid according to one of claims 1 to 4 and/or a fragment according to one of claims 5 or 6 in a plant cell; and
regeneration of a plant and/or plant cell and/or plant tissue and/or plant organ from the transformed plant cell.

19. Process for influencing the allene oxide cyclase activity of a plant cell, of seed and/or of a plant tissue and/or plant organ and/or of a plant comprising the step:
introduction of a nucleic acid according to one of claims 1 to 4 and/or of a fragment according to one of claims 5 or 6 in a plant cell and/or in a seed and/or in a plant tissue and/or plant organ and/or in a plant.

20. Process for the selective production of 9S/13S (cis(+))-12-oxophytodienic acid in jasmonic acid biosynthesis comprising the following steps:
introduction and expression of a nucleic acid according to one of claims 1 to 4 in a suitable host cell.
introduction and expression of a nucleic acid according to one of claims 1 to 4 together with nucleic acids, coding for lipoxygenases and allene oxide synthases, in a suitable host cell.

21. Process for the production of jasmonic acid comprising the step:
production of the precursor molecule 9S/13S (cis(+))-12-oxophytodienic acid according to claim 20.

22. Use of a plant cell according to claim 11 for the production of whole plants.

23. Use of a nucleic acid according to one of claims 1 to 4 for the isolation of homologous sequences from plants.

24. Use of a nucleic acid according to one of claims 1 to 4 for the expression of an allene oxide cyclase in pro- and/or eukaryotic cells.

25. Use of a nucleic acid according to one of claims 1 to 4 under the control of a regulatory element in antisense orientation for inhibition of the expression of an allene oxide cyclase in pro- and/or eukaryotic cells.

26. Use of a nucleic acid according to one of claims 1 to 4 for the production of transgenic crop plants with modified characteristics, with the modified characteristics being selected in particular from:
a) increased pathogen defence;
b) increased defence against herbivores;
c) optimised plant-useful insect-pest interaction;
d) increased biomass formation;
e) modified carbohydrate balance;
f) modified nitrogen balance;
g) increased formation of secondary natural substances, particularly alkaloids and/or phytoalexins;
h) optimised UV protection;
i) modified male sterility;
l) modified developmental processes, particularly in flower development and/or seed formation and/or germination.
